(19) United States
(12) Patent Application Publication
(10) Pub. No.: US 2025/0389686 A1
(43) Pub. Date: Dec. 25, 2025

(54) BIOLOGICAL INFORMATION MEASUREMENT DEVICE AND BIOLOGICAL INFORMATION MEASUREMENT SYSTEM

(71) Applicant: OMRON HEALTHCARE Co., Ltd., Kyoto (JP)

(72) Inventors: Kenji ONO, Kyoto (JP); Kazuma YASUDA, Kyoto (JP); Kosuke INOUE, Kyoto (JP); Asa ANAI, Kyoto (JP); Takashi YASUDA, Kyoto (JP)

(21) Appl. No.: 19/305,319

(22) Filed: Aug. 20, 2025

Related U.S. Application Data
(63) Continuation of application No. PCT/JP2023/040736, filed on Nov. 13, 2023.

(30) Foreign Application Priority Data
Mar. 30, 2023 (JP) 2023-056661

(51) Int. Cl. G01N 27/327; G01N 27/30

(52) U.S. Cl. CPC G01N 27/327; G01N 27/301

(57) ABSTRACT

A biological information measurement system including a biological information measurement device and an information processing terminal configured to communicate with the biological information measurement device, the biological information measurement system provided with a first electrode, a second electrode, and a third electrode, and configured to measure biological information of a measurement target based on a potential difference between the first electrode and the second electrode with a potential of the third electrode as a reference potential, in which the biological information measurement device includes a first contact signal output means, a second contact signal output means, a contact state classification unit configured to classify a level of the contact state of each of the first electrode and the second electrode with respect to the measurement target into at least three stages based on each signal output from the first converter and the second converter.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040736, filed Nov. 13, 2023, which application claims priority to Japanese Patent Application No. 2023-056661, filed Mar. 30, 2023, which applications are incorporated herein by reference in their entireties.

### TECHNICAL FIELD

This invention relates to the health care-related technical field, and in particular, relates to a biological information measurement device and a biological information measurement system.

### BACKGROUND

In recent years, health management is increasingly popular to manage health by measuring information about an individual's body and health (hereinafter referred to as “biological information”), such as blood pressure values and electrocardiograms, using a measurement device, and recording and analyzing the measurement results on an information processing terminal.

As an example of the measurement device described above, a portable electrocardiographic measurement device (for example, Patent Document 1) has been proposed to immediately measure electrocardiograms when abnormalities such as chest pain or palpitations occur in daily life, and is expected to contribute to early detection and appropriate treatment of cardiac diseases.

Patent Document 1 discloses a portable electrocardiogram recording device that measures and records an electrocardiographic waveform by a pair of electrodes that are placed in contact with the right hand and the skin of the chest, in which an electric circuit detects whether the contact resistance between the skin and the electrode is sufficiently small, and if not, the contact failure is reported to the measurer by display, sound, or the like.

According to such a technique, when the contact resistance between the skin and the electrode is not sufficiently small (that is, when the electrode contact state is not good enough for the normal measurement), the state can be reported to the measurer who can record a normal electrocardiogram after taking measures such as reattaching the electrode or applying water.

### CITATION LIST

### Patent Literature

Patent Document 1: JP H10-234689 A

### SUMMARY OF INVENTION

### Technical Problem

Even when the technique described in Patent Document 1 is employed, there is a problem that the measurer can only recognize whether the contact state of the electrode is poor or not, and thus cannot tell how much the electrode should be pressed against the skin. This may cause an excessive force to be applied to press the electrode against the skin more than necessary and superimpose a myoelectric noise on the electrocardiographic signal, thereby preventing correct recording of the electrocardiogram.

In view of the conventional technique described above, it is an object of the present invention to provide a technique to report a contact state of an electrode with respect to a measurement target by level in three or more stages when measuring biological information using a biological information measurement device including the electrode.

### Solution to Problem

In an aspect, the present invention employs the following configuration to solve the above-described problems. Specifically, a biological information measurement device provided with a first electrode, a second electrode, and a third electrode, and configured to measure biological information of a measurement target based on a potential difference between the first electrode and the second electrode with a potential of the third electrode as a reference potential, the biological information measurement device including
a first contact signal output means configured to output a signal related to a contact state of the first electrode with respect to the measurement target based on a potential of the first electrode that varies depending on a contact state of the first electrode with respect to the measurement target,
a second contact signal output means configured to output a signal related to a contact state of the second electrode with respect to the measurement target based on a potential of the second electrode that varies depending on a contact state of the second electrode with respect to the measurement target,
a contact state classification means configured to classify a level of the contact state of each of the first electrode and the second electrode with respect to the measurement target into at least three stages based on a signal output from each of the first contact detection means and the second contact detection means,
a reporting means configured to report the contact state of each of the first electrode and the second electrode with respect to the measurement target in a manner that the level classified by the contact state classification means is identifiable, and
a control means configured to execute processing of measuring the biological information.

According to such a configuration, the contact state of the first electrode and the second electrode with respect to the contact target can be indicated by a level of three or more stages, so that the user can intuitively grasp how much each electrode should be pressed against the skin depending on the current level of the contact state of the electrode.

The reporting means may include a sound output means to allow reporting by sound. The reporting means may include a vibration means to allow reporting by vibration. The reporting means may include a display means to allow reporting by display. Since there are many different ways in which the information on the contact state should be perceived and acquired depending on the environment in which the device is used, the characteristics of the user, and the timing of the report, it is desirable to allow reporting of the information by various output methods.

The reporting means may indicate the level by displaying at least one of a numerical value, a number of a plurality of display segments whose display is activated, a size of an area whose display is activated, or a change in color and transparency of a display area in the display means. This allows the user to easily grasp the level of the contact state of each electrode with respect to the skin.

The reporting means may report the contact state of each of the first electrode and the second electrode with respect to the measurement target before and/or during the measurement processing of the biological information. By grasping the level of the contact state not only before the start of the measurement but also during the measurement, the user can more stably maintain a good contact state and perform accurate measurement.

The present invention can also be understood as a biological information measurement system as below. Specifically, a biological information measurement system including a biological information measurement device and an information processing terminal that communicates with the biological information measurement device, the biological information measurement device being provided with a first electrode, a second electrode, and a third electrode, and configured to measure biological information of the measurement target based on a potential difference between the first electrode and the second electrode with a potential of the third electrode as a reference potential,
in which the biological information measurement device includes
a control means configured to execute processing of measuring the biological information,
a first contact detection means configured to output a signal related to a contact state of the first electrode with respect to the measurement target based on a potential of the first electrode that varies depending on a contact state of the first electrode with respect to the measurement target, and
a second contact detection means configured to output a signal related to a contact state of the second electrode with respect to the measurement target based on a potential of the second electrode that varies depending on a contact state of the second electrode with respect to the measurement target,
at least the biological information measurement device or the information processing terminal includes
a contact state classification means configured to classify a level of the contact state of each of the first electrode and the second electrode with respect to the measurement target into at least three stages based on a signal output from each of the first contact detection means and the second contact detection means, and
the information processing terminal includes
a reporting means configured to report a contact state of each of the first electrode and the second electrode with respect to the measurement target in a manner that the level classified by the contact state classification means is identifiable.

Thus, by providing the means for reporting the contact state level of the electrode with respect to the measurement target as a separate terminal, the degree of freedom in the manner of reporting can be increased, and more usable reporting can be performed.

In the biological information measurement system, the reporting means may include a display means to allow reporting by display. The reporting means may indicate the level by displaying at least one of a numerical value, a number of a plurality of display segments whose display is activated, a size of an area whose display is activated, or a change in color and transparency of a display area in the display means. The reporting means may report the contact state of each of the first electrode and the second electrode with respect to the measurement target before and/or during the measurement processing of the biological information.

The above configurations and processing can be combined with each other to form the present invention as long as no technical contradiction arises.

### Advantageous Effects of Invention

According to the present invention, when the biological information is measured using the biological information measurement device including the electrodes, the contact state of the electrodes with respect to the measurement target can be reported by a level in three or more stages.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic diagram for schematically explaining a biological information measurement system according to a first embodiment;

FIG. 2(A) is a front view illustrating the configuration of a portable electrocardiograph according to the first embodiment;

FIG. 2(B) is a rear view illustrating the configuration of the portable electrocardiograph according to the first embodiment;

FIG. 2(C) is a left side view illustrating the configuration of the portable electrocardiograph according to the first embodiment;

FIG. 2(D) is a right side view illustrating the configuration of the portable electrocardiograph according to the first embodiment;

FIG. 2(E) is a plan view illustrating the configuration of the portable electrocardiograph according to the first embodiment;

FIG. 2(F) is a bottom view illustrating the configuration of the portable electrocardiograph according to the first embodiment;

FIG. 3 is a circuit diagram schematically illustrating an electric circuit configuration including electrodes of the portable electrocardiograph according to the first embodiment;

FIG. 4 is a flowchart illustrating a part of a processing flow of a portable electrocardiograph and a smartphone, respectively, when they are connected for communication in the biological information measurement system according to the first embodiment;

FIG. 5 is a flowchart illustrating a part of the processing flow of the portable electrocardiograph and the smartphone, respectively, when they are connected for communication in the biological information measurement system according to the first embodiment;

FIG. 6 is a flowchart illustrating a part of the processing flow of the portable electrocardiograph and the smartphone, respectively, when they are connected for communication in the biological information measurement system according to the first embodiment;

FIG. 7 is a flowchart illustrating a subroutine of a processing when BLE communication is performed by the portable electrocardiograph according to the first embodiment;

FIG. 8(A) is a first view of a display example illustrating an electrode contact level in the biological information measurement system according to the first embodiment;

FIG. 8(B) is a second view of the display example illustrating the electrode contact level in the biological information measurement system according to the first embodiment;

FIG. 8(C) is a third view of the display example illustrating the electrode contact level in the biological information measurement system according to the first embodiment;

FIG. 9(A) is a first view of an example of a screen displayed on a smartphone during measurement of the electrocardiogram in the biological information measurement system according to the first embodiment;

FIG. 9(B) is a second view of the example of the screen displayed on the smartphone during measurement of the electrocardiogram in the biological information measurement system according to the first embodiment;

FIG. 9(C) is a third view of the example of the screen displayed on the smartphone during measurement of the electrocardiogram in the biological information measurement system according to the first embodiment;

FIG. 9(D) is a fourth view of the example of the screen displayed on the smartphone during measurement of the electrocardiogram in the biological information measurement system according to the first embodiment;

FIG. 10(A) is a first view of a modified example of a display illustrating the electrode contact level in the information measurement system according to the first embodiment;

FIG. 10(B) is a second view of the modified example of the display illustrating the electrode contact level in the information measurement system according to the first embodiment;

FIG. 10(C) is a third view of the modified example of the display illustrating the electrode contact level in the information measurement system according to the first embodiment;

FIG. 11(A) is a front view illustrating the configuration of a portable electrocardiograph according to a second embodiment;

FIG. 11(B) is a rear view illustrating the configuration of the portable electrocardiograph according to the second embodiment;

FIG. 11(C) is a left side view illustrating the configuration of the portable electrocardiograph according to the second embodiment;

FIG. 11(D) is a right side view illustrating the configuration of the portable electrocardiograph according to the second embodiment;

FIG. 11(E) is a plan view illustrating the configuration of the portable electrocardiograph according to the second embodiment;

FIG. 11(F) is a bottom view illustrating the configuration of the portable electrocardiograph according to the second embodiment;

FIG. 12 is a block diagram illustrating a functional configuration of the portable electrocardiograph according to the second embodiment; and,

FIG. 13 is a flowchart illustrating a flow of an electrocardiographic waveform measurement processing by the portable electrocardiograph device according to the second embodiment.

### DESCRIPTION OF EMBODIMENTS

### First Embodiment

Embodiments of the present invention will be specifically described below with reference to the drawings. It should be noted that the dimensions, material, shape, relative arrangement and the like of the constituent components described in the embodiments are not intended to limit the scope of the present invention to those alone, unless otherwise stated.

### System Configuration

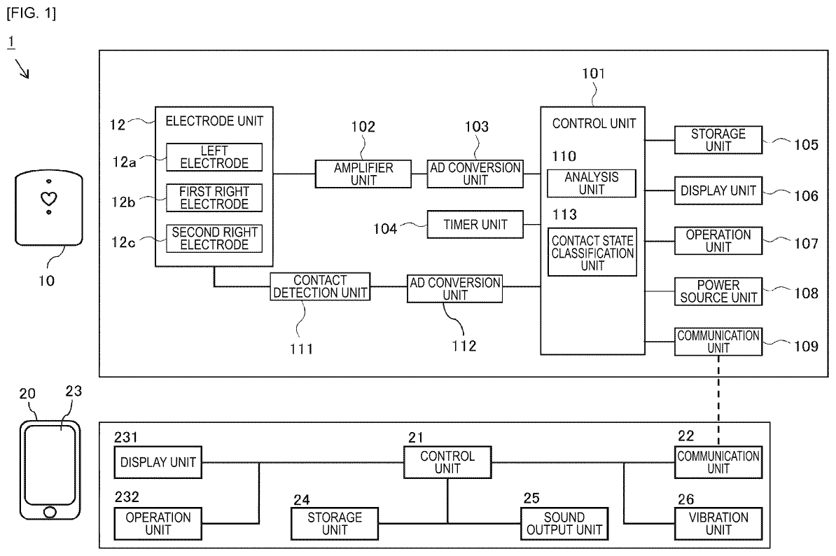

FIG. 1 is a schematic diagram illustrating a configuration example of a biological information measurement system 1 according to the present embodiment. As illustrated in FIG. 1, the biological information measurement system 1 includes a portable electrocardiograph 10 as an example of a biological information measurement device and a smartphone 20 as an example of an information processing terminal, and these are configured to be connectible and communicable to each other.

### Portable Electrocardiograph

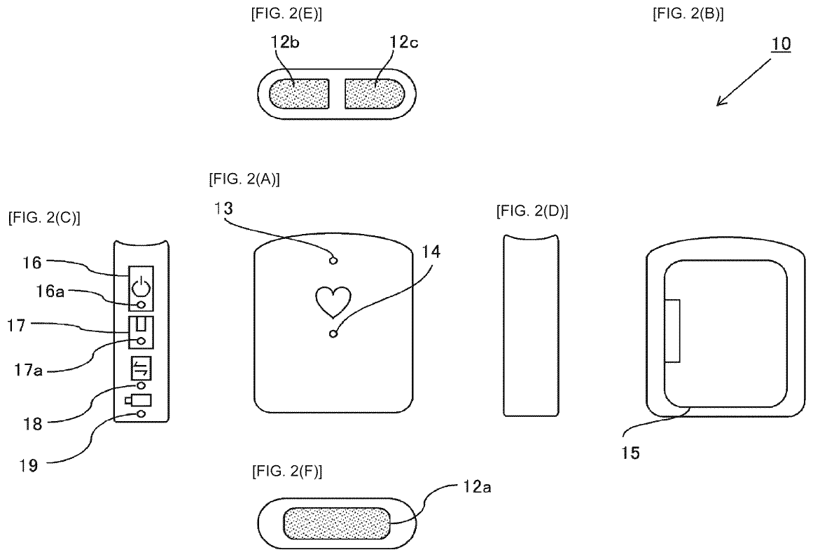

FIGS. 2(A) to 2(F) are collectively a diagram illustrating a configuration of the portable electrocardiograph 10 according to the present embodiment. FIG. 2(A) is a front view illustrating a front surface of the main body. Similarly, FIG. 2(B) is a rear view, FIG. 2(C) is a left side view, FIG. 2(D) is a right side view, FIG. 2(E) is a plan view, and FIG. 2(F) is a bottom view.

On the bottom surface of the portable electrocardiograph 10, a left electrode 12a that is placed in contact with the left side of the body during electrocardiographic measurement is provided. On the upper surface side of the portable electrocardiograph 10 opposite to the bottom surface, a first right electrode 12b that is similarly placed in contact with the middle section of the right index finger and a second right electrode 12c that is placed in contact with the base section of the right index finger are provided. The first right electrode 12b functions as a GND electrode.

During electrocardiographic measurement, the user holds the portable electrocardiograph 10 by the right hand, and places the right index finger on the upper surface portion of the portable electrocardiograph 10 in proper contact with the first right electrode 12b and the second right electrode 12c. In this state, the left electrode 12a is placed in contact with the skin at a location corresponding to a desired measurement method. For example, when a so-called lead I measurement is performed, the left electrode 12a is placed in contact with the palm of the left hand, and when a so-called lead V4 measurement is performed, the left electrode 12a is placed in contact with the skin of the left chest slightly to the left of the epigastric region and below the nipple.

Various operation units and indicators are disposed on the left side surface of the portable electrocardiograph 10. Specifically, a power source switch 16, a power source LED 16a, a Bluetooth (trade name) Low Energy (BLE) communication button 17, a BLE communication LED 17a, a memory residual indicator LED 18, a battery exchange LED 19, and the like, are provided.

On the front surface of the electrocardiograph 10, a measurement state reporting LED 13 and an analysis result reporting LED 14 are provided. On the rear surface of the electrocardiograph 10, a battery housing opening and a battery cover 15 are disposed.

FIG. 1 is a block diagram illustrating a functional configuration of the portable electrocardiograph 10. As illustrated in FIG. 1, the portable electrocardiograph 10 includes functional units consisting of a control unit 101, an electrode unit 12, an amplifier unit 102, an analog to digital (AD) conversion unit 103, a timer unit 104, a storage unit 105, a display unit 106, an operation unit 107, a power source unit 108, a communication unit 109, a contact detection unit 111, and an AD conversion unit 112.

The control unit 101 is a means for controlling the portable electrocardiograph 10, and includes a central processing unit (CPU) and the like, for example. Upon receipt of an operation by a user via the operation unit 107, the control unit 101 controls the constituent components of the portable electrocardiograph 10 to execute various types of processing such as electrocardiographic measurement and information communication in accordance with predetermined recording mediums. The predetermined recording mediums are stored in the storage unit 105 which will be described later and read therefrom.

The control unit 101 includes, as functional modules, an analysis unit 110 that analyzes electrocardiographic waveforms and a contact state classification unit 113. The analysis unit 110 analyzes the measured electrocardiogram to determine whether there is any disturbance in the waveform, and outputs the result as to whether the electrocardiogram is normal at least at the time of measurement. The contact state classification unit 113 classifies the levels of the contact state of the left electrode 12a and the first right electrode 12b detected by the contact detection unit 111 into four stages. The detection of the contact state and the classification of the levels thereof will be described later.

The electrode unit 12 consists of the left electrode 12a, the first right electrode 12b, and the second right electrode 12c, and functions as a sensor for detecting the electrocardiographic waveform. Specifically, the second right electrode 12c is used as a ground (GND) electrode and, with respect to this reference potential, the potential difference between the potential of the left electrode 12a and the potential of the first right electrode 12b is continuously measured to acquire the electrocardiographic waveform. A specific circuit configuration for the electrocardiographic waveform detection will be described later.

The amplifier unit 102 has a function of amplifying a signal indicating the electrocardiographic waveform output from the electrode unit 12 as described later. The AD conversion unit 103 has a function of converting an analog signal amplified by the amplifier unit 102 into a digital signal and transmitting the converted signal to the control unit 101.

The timer unit 104 has a function of measuring time with reference to a real time clock (RTC). For example, as will be described later, when the electrode contact detection processing is performed, the time during which all of the left electrode 12a, the first right electrode 12b, and the second right electrode 12c are in contact with the body is counted. Alternatively, the time to the end of measurement during the electrocardiographic measurement may be counted and output.

The storage unit 105 includes a main storage device such as a random access memory (RAM) or a read only memory (ROM), and stores various kinds of information such as application recording mediums, measured electrocardiographic waveforms, and analysis results. In addition to the RAM or the ROM, the storage unit 105 includes a long-term storage medium such as a flash memory.

The display unit 106 includes the measurement state reporting LED 13, the analysis result reporting LED 14, the power source LED 16a, the BLE communication LED 17a, the memory residual indicator LED 18, the battery exchange LED 19, and the like, and transmits the state of the device to the user by turning on or blinking the LEDs. The operation unit 107 includes the power source switch 16, the communication button 17, and the like, and has a function of receiving an input operation from the user and causing the control unit 101 to execute processing corresponding to the operation.

The power source unit 108 includes a battery that supplies power required for operation of the device. The battery may be, for example, a secondary battery such as a lithium ion battery, or a primary battery.

The communication unit 109 includes an antenna for wireless communication, and has a function of communicating, at least by the BLE communication, with other devices such as an information processing terminal which will be described later. A terminal for wired communication may also be provided.

The contact detection unit 111 includes an electric circuit connected to the left electrode 12a and the first right electrode 12b, thereby detecting the contact state of the skin surface of the measurement target with respect to the left electrode 12a and the first right electrode 12b, and outputting a signal corresponding to the contact state according to the level of the contact state. The AD conversion unit 112 converts an analog signal output from the contact detection unit 111 to a digital signal and transmits it to the control unit 101.

### Electric Circuit Configuration

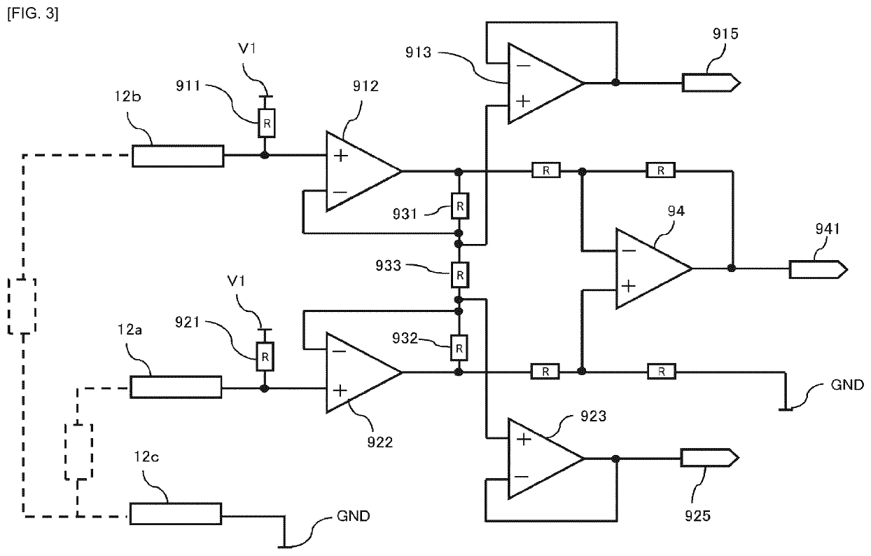

The contact state detection and the electrocardiographic waveform measurement in the portable electrocardiograph 10 according to the present embodiment will be described below with reference to FIG. 3. FIG. 3 is a circuit diagram schematically illustrating an electric circuit including the electrodes of the portable electrocardiograph 10.

As illustrated in FIG. 3, the second right electrode 12c is connected to the reference potential GND and functions as the ground terminal. The first right electrode 12b is connected to the power source potential V1 via a right pull-up resistor 911. The left electrode 12a is connected to the power source potential V1 via a left pull-up resistor 921. The power source potential V1 is set to a potential (for example, 4V) which is higher than the reference potential GND and can secure a sufficient bias.

Therefore, when the power source is turned on and both the first right electrode 12b and the second right electrode 12c are correctly placed in contact with the body skin, a current flows to the second right electrode 12c having a potential lower than the first right electrode 12b via the impedance of the human body, thereby changing the potential of the first right electrode 12b. Such a change in potential depends on the contact state of the first right electrode 12b (and the second right electrode 12c) with respect to the skin surface.

That is, the more firmly the first right electrode 12b is in contact with the skin, the lower the potential, so that the contact state of the first right electrode 12b with respect to the skin can be determined based on the potential. The same applies to the left electrode 12a. In FIG. 3, the circuit indicated by a dashed line portion indicates the current path through the impedance of the human body.

The right pull-up resistor 911 and the left pull-up resistor 921 are set to sufficiently high resistance values (for example, 200 MΩ, preferably 300 MΩ or greater) in order to secure the accuracy of the detected electrocardiographic waveform.

Arranged in the circuit illustrated in FIG. 3 are five amplifiers including a right non-inverting amplifier 912, a right buffer amplifier 913, a left non-inverting amplifier 922, a left buffer amplifier 923, and a differential amplifier 94.

As illustrated in FIG. 3, the potential of the first right electrode 12b is input to the positive input terminal of the right non-inverting amplifier 912. A right amplified signal amplified by the amplification factor defined by a first amplification factor determining resistor 931 and a third amplification factor determining resistor 933 is output from the output terminal of the right non-inverting amplifier 912 and input to the negative terminal of the differential amplifier 94. A signal having the same potential as that input to the positive input terminal of the right non-inverting amplifier 912 is input to the positive input terminal of the right buffer amplifier 913 via the right non-inverting amplifier 912. That is, the right non-inverting amplifier 912 functions as a normal amplifier (signal amplifier) and also functions as a buffer (voltage follower).

The right buffer amplifier 913 functions as a buffer, and a signal having the same potential as the potential input to the positive input terminal is output from the output terminal. The output signal is input to the AD conversion unit 112 as a right contact state signal 915, converted into a digital signal, and transmitted to the control unit 101.

The potential of the left electrode 12a is input to the positive input terminal of the left non-inverting amplifier 922. A left amplified signal amplified by the amplification factor defined by a second amplification factor determining resistor 932 and the third amplification factor determining resistor 933 is output from the output terminal of the left non-inverting amplifier 922 and input to the positive terminal of the differential amplifier 94. A signal having the same potential as that input to the positive input terminal of the left non-inverting amplifier 922 is input to the positive input terminal of the left buffer amplifier 923 via the left non-inverting amplifier 922. That is, similar to the right non-inverting amplifier 912, the left non-inverting amplifier 922 also functions as a normal amplifier while functioning as a buffer. The resistance values of the first amplification factor determining resistor 931 and the second amplification factor determining resistor 932 are set to the same value.

The left buffer amplifier 923 functions as a buffer, and a signal having the same potential as the potential input to the positive terminal is output from the output terminal. The output signal is input to the AD conversion unit 112 as the left contact state signal 925, converted into a digital signal, and transmitted to the control unit 101.

The contact state classification unit 113, which is a functional module of the control unit 101, classifies the levels of the contact state of each of the first right electrode 12b and the left electrode 12a to the skin into four stages of “good contact”, “slightly poor contact”, “poor contact”, and “no contact” using the right contact state signal 915 and the left contact state signal 925 which have been digitally converted by the AD conversion unit 112. The user can appropriately set a threshold value for classifying the digitized signals based on the contact resistance, the quality of the electrocardiographic record, and the like. The classified level of the contact state is stored in the storage unit 105. Since the contact state classification can change over time by reflecting the change of the right contact state signal 915 and left side contact state signal 925 as they change over time, information indicating the level of the classified contact state is recorded in the storage unit 105 as time series data.

The differential amplifier 94 is a differential amplifier that amplifies and outputs the difference between the potential of the first right electrode 12b input to the negative input terminal thereof after being amplified and output by the right non-inverting amplifier 912 and the potential of the left electrode 12a input to the positive input terminal thereof after being amplified and output by the left non-inverting amplifier 922. That is, the differential amplifier 94 is included in the amplifier unit 102, and the signal output from the differential amplifier 94 is the electrocardiographic signal of the measurement target. The electrocardiographic signal is further input to the AD converter 103, and the signal converted into a digital signal is transmitted to the control unit 101 and recorded as the electrocardiographic waveform in the storage unit 105 by the control unit 101.

### Smartphone

The information processing terminal may be, for example, a smartphone 20 including a touch panel display 23. As illustrated in FIG. 1, the smartphone 20 includes functional units including a control unit 21, a communication unit 22, a display unit 231, an operation unit 232, a storage unit 24, a sound output unit 25, and a vibration unit 26.

The control unit 21 is a control means of the smartphone 20 and includes, for example, a CPU, thereby performing functions corresponding to various recording mediums stored in the storage unit 24 by executing such recording mediums. The communication unit 22 includes an antenna for wireless communication, and is a function of communicating with other devices such as the portable electrocardiograph 10 and a wireless base station. A terminal for wired communication may also be included.

The display unit 231 includes a touch panel display 23 on which various types of information are displayed. As described later, when a communication connection with the portable electrocardiograph 10 is established, the touch panel display 23 can display the classified contact state level and the like transmitted from the portable electrocardiograph 10. That is, this is an example of a reporting means. The operation unit 232 includes the touch panel display 23 and receives various inputs from the user via an image for input.

The storage unit 24 includes, for example, the long-term storage medium such as the flash memory, in addition to the main memory such as a RAM, and stores various types of information such as application recording mediums, measured electrocardiographic waveforms, and analysis results.

The sound output unit 25 includes a speaker not illustrated, and can also report the classified contact state level transmitted from the portable electrocardiograph 10 by sound when the communication connection with the portable electrocardiograph 10 is established as will be described later.

The vibration unit 26 includes a vibrator not illustrated, and can report the classified contact state level transmitted from the portable electrocardiograph 10 by vibration (and by pattern) when the communication connection with the portable electrocardiograph 10 is established as will be described later.

### Measurement Processing Flow in System

The portable electrocardiograph 10 can perform the electrocardiographic measurement, the analysis of the measurement data, and the display of the analysis result by itself, but can also be used by establishing the communication connection with the information processing terminal to improve convenience. Hereinafter, a case in which the portable electrocardiograph 10 is used by establishing the communication connection with the smartphone 20 will be described.

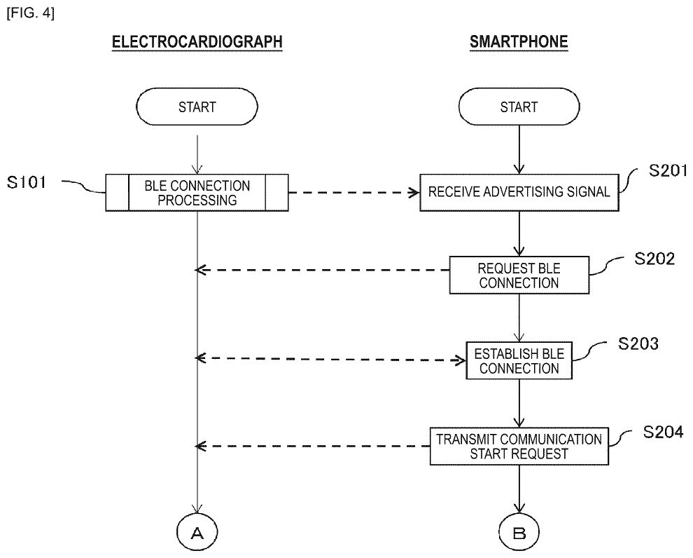

FIGS. 4, 5, and 6 are timing diagrams illustrating processing flows and information transmission timing between the devices when the portable electrocardiograph 10 and the smartphone 20 are linked by the BLE communication to perform the electrocardiographic measurement.

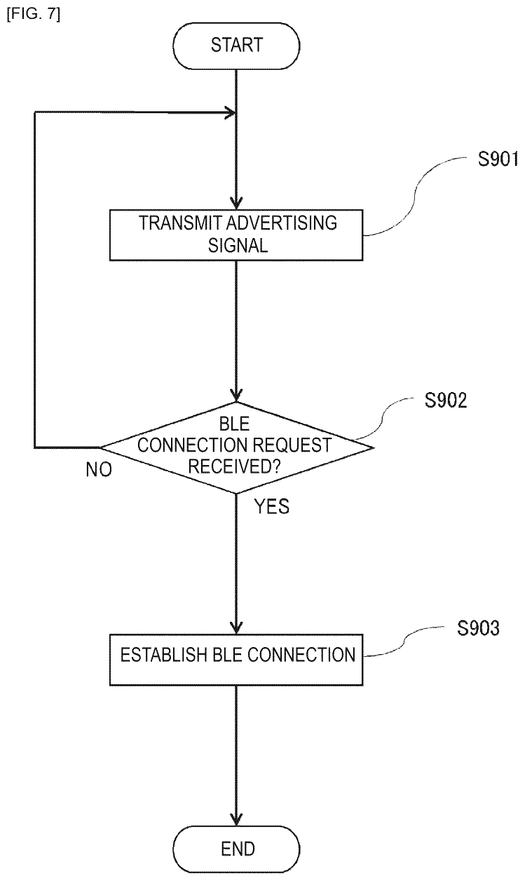

First, when the user operates the power source switch 16 of the portable electrocardiograph 10 to turn on the power source, a subroutine processing for the BLE communication is executed in the portable electrocardiograph 10 (S101), as illustrated in FIG. 4. FIG. 7 is a flowchart illustrating a flow of the subroutine processing. When the power source is turned on, the control unit 101 of the portable electrocardiograph 10 transmits an advertising signal for BLE communication from the communication unit 109 (S901). Subsequently, the control unit 101 determines whether the connection request for the BLE communication is received from a different information processing terminal (S902). If it is determined that no connection request for the BLE communication has been received, similar processing steps are repeated until the processing of the BLE communication is canceled by the elapse of predetermined time or by the operation of the operation unit 107. If it is determined that the connection request for BLE communication has been received, the process proceeds to step S903 to establish the BLE connection with the device having transmitted the connection request. When the BLE communication connection is established, the control unit 101 ends the subroutine. A start trigger of the subroutine is not limited to the turn-on of the power source, and may be initiated by, for example, operating the BLE communication button 17.

The user places the smartphone 20 in a state allowing the BLE communication with the portable electrocardiograph 10. Specifically, the user operates the touch panel display 23 to turn on the BLE connection settings in a setting menu or the like. Alternatively, the BLE connection settings may be turned on by initiating a dedicated application recording medium to link with the portable electrocardiograph 10.

By referring to FIG. 4 again, when the BLE connection settings are turned on, the control unit 21 of the smartphone 20 receives the advertising signal for the BLE communication via the communication unit 22 (S201), and transmits the BLE connection request to the portable electrocardiograph 10 (S202). Subsequently, the BLE connection is established with the portable electrocardiograph 10 (S203 which corresponds to S903 described above), and the communication start request is transmitted (S204).

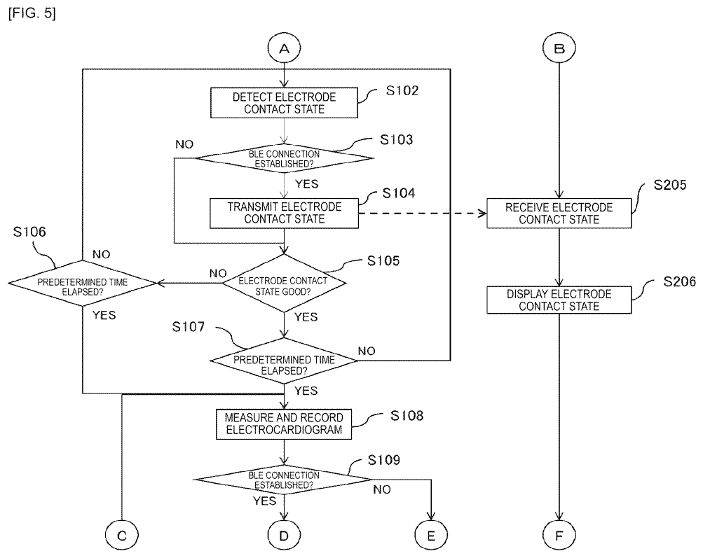

To measure the electrocardiogram, the user holds the portable electrocardiograph 10 with the right hand, and places the right index finger in contact with the first right electrode 12b and the second right electrode 12c, while placing the left electrode 12a in contact with the skin at a location to be measured. As illustrated in FIG. 5, when the subroutine for the BLE communication ends, the portable electrocardiograph 10 detects the contact state of the first right electrode 12b and the left electrode 12a with respect to the skin, and classifies the levels of the detected contact state into the four stages described above (S102). Subsequently, the portable electrocardiograph 10 determines whether the BLE connection is established (S103). If it is determined here that the BLE connection is established, the information indicating the (classified) level of the electrode contact state is transmitted to the smartphone 20 (S104), and the information is received by the smartphone 20 (S205). If it is determined that the BLE connection is not established in step S103 in the electrocardiograph 10, the process skips step S104 and proceeds to S105 in which it is determined whether the electrode contact state is “good”.

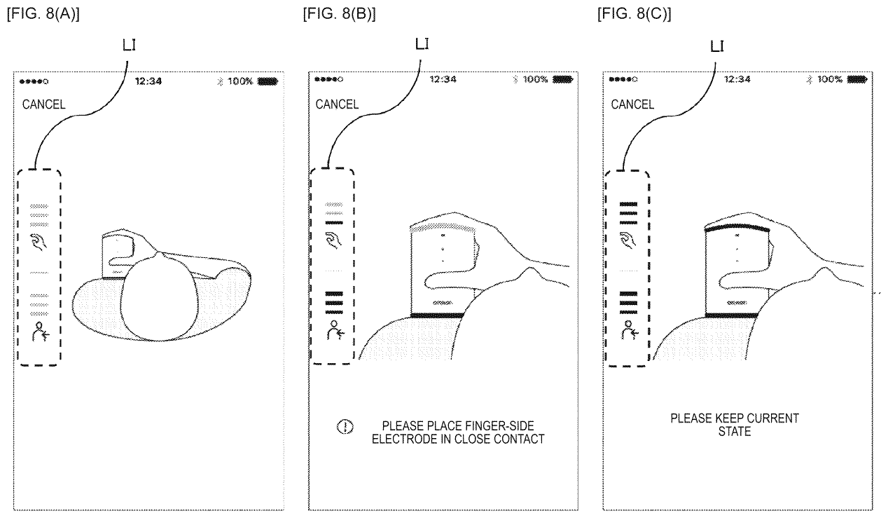

Upon receipt of the information of the electrode contact state, the smartphone 20 displays the information indicating the level of the electrode contact state on the touch panel display 23 (S206). FIGS. 8(A) to 8(C) illustrate display examples of the touch panel display 23 including information indicating the level of the electrode contact state. As illustrated in FIGS. 8(A) to 8(C), the screen displays an image simulating the user performing the electrocardiographic measurement by the lead IV method with the portable electrocardiograph 10, while displaying an electrode contact level indicator LI which is an area on the left side of the screen for indicating the level of the electrode contact state. The electrode contact level indicator LI is configured to display three display segments on the upper side, indicating the contact levels of the first right electrode 12b, and three display segments on the lower side, indicating the contact level of the left electrode 12a.

The contact state levels of each electrode are indicated by the number of display segments whose display is activated among the three display segments. When the number of segments whose display is activated is zero, it means “no contact”, and when all three display segments are activated, it means “good contact”. When the display of one segment is activated, it means “poor contact”, and when the display of two segments is activated, it means “slightly poor contact”.

As illustrated in FIG. 8(B), the electrode contact level indicator LI indicates the contact level of the first right electrode 12b and the contact level of the left electrode 12a separately. In the example of FIG. 8(B), the contact level of the first right electrode 12b is indicated as “poor contact”, and the contact level of the left electrode 12a is indicated as “good”. In the example of FIG. 8(C), the contact levels of both electrodes are indicated as “good”.

In addition to the electrode contact level indicator LI, the touch panel display 23 may also display information for advising the user on how to (maintain) the “good” contact state. For example, when the first right electrode 12b is in the “slightly poor contact” state as illustrated in FIG. 8(B), “Please place the finger-side electrode in close contact” may be displayed, or when both electrodes are in the “good contact” state as illustrated in FIG. 8(C), “Please keep the current state” may be displayed.

By referring to FIG. 5 again, in the processing of step S105, the portable electrocardiograph 10 performs processing of determining whether the contact state of both the first right electrode 12b and the left electrode 12a is “good” (S105). If it is determined that the contact state of at least one of the electrodes is not “good”, the process proceeds to step S106 in which it is determined whether predetermined time has elapsed in that state (S106). The predetermined time here is set to a reasonable amount of time (for example, five seconds) for waiting to allow the user to make the electrode contact state be “good”. If it is determined in the portable electrocardiograph 10 that the predetermined time has not elapsed in step S106, the process returns to step S102, and the subsequent processing steps are repeated. In step S106, if it is determined that the predetermined time has elapsed, the process proceeds to step S108 in the portable electrocardiograph 10.

If it is determined in the portable electrocardiograph 10 that the contact states of both electrodes are “good” in step S105, the processing of determining whether predetermined time has elapsed in that state (S107). The predetermined time here is set to a reasonable amount of time (for example, three seconds) for determining whether the “good” contact state is stable rather than transient for both electrodes. If it is determined in the electrocardiograph 10 that the predetermined time has not elapsed in step S107, the process returns to step S102 and the subsequent processing steps are repeated. If it is determined in step S107 that the predetermined time has elapsed, the process proceeds to step S108.

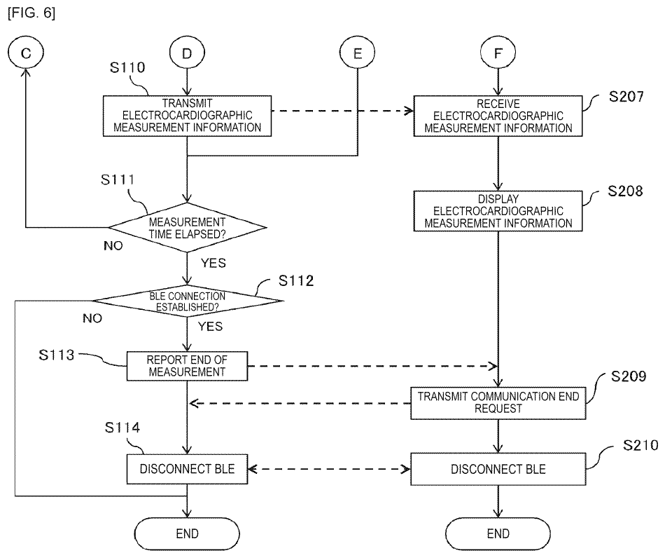

In step S108, the portable electrocardiograph 10 executes the electrocardiographic measurement processing to measure and record the electrocardiographic waveform (S108). Specifically, the processing of storing the electrocardiographic signal in the storage unit 105 as needed is performed, in which the electrocardiographic signal is output from the differential amplifier 94 and input to the control unit 101 via the AD conversion unit 103. Subsequent to (actually, in parallel to) step S108, the portable electrocardiograph 10 performs processing of determining whether the BLE connection is established (S109). If it is determined here that the BLE connection is established, as illustrated in FIG. 6, the information related to the electrocardiographic measurement, such as the measured electrocardiogram, the electrode contact state, and the elapsed time from the start of measurement (or remaining time to the end of measurement) is transmitted to the smartphone 20 (S110), and the information is received by the smartphone 20 (S207). Subsequent to step S110, the portable electrocardiograph 10 executes processing of determining whether predetermined time (for example, 30 seconds) for the electrocardiographic measurement has elapsed (S111). If it is determined in the portable electrocardiograph 10 that the BLE connection is not established in step S109, the process skips step S110 and proceeds to S111.

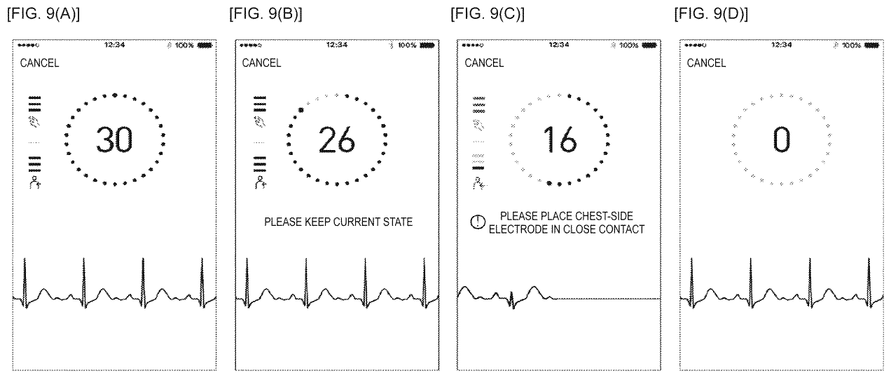

The smartphone 20 that has received the information on the electrocardiographic measurement displays the information on the touch panel display 23 (S208). FIGS. 9(A) to 9(D) illustrate examples of screens displayed on the touch panel display 23 during execution of the electrocardiographic measurement processing. As illustrated in FIGS. 9(A) to 9(D), when the BLE communication connection is established, the touch panel display displays the number indicating the remaining time (seconds) of the measurement, the electrocardiographic waveform, and the electrode contact level indicator LI indicating the level of the electrode contact state. The display of the number of seconds is counted down in accordance with the elapse of the measurement time, and the display segments arranged in a circular shape around the number of seconds are gradually deactivated. The electrode contact level indicator LI also indicates the contact state of the electrode with respect to the skin in real time. As illustrated in FIG. 9(C), as the electrode contact state deteriorates, the number of segments whose display is activated decreases, and a message (Please place the chest-side electrode in close contact) is also displayed to urge the user to correct the electrode contact state. When the acquired electrocardiographic waveform is disturbed, the disturbance is also reflected in the display of the touch panel display 23.

In the portable electrocardiograph 10, it is determined whether the predetermined time for the electrocardiographic measurement has elapsed in step S111. When it is determined that the predetermined time has not elapsed, the process returns to step S108 and the subsequent processing steps are repeated. When it is determined in step S111 that the predetermined measurement time has elapsed, the processing of determining whether the BLE connection is established is executed (S112). When it is determined in the portable electrocardiograph 10 that the BLE connection is established in step S112, the series of processing steps end as they are. When it is determined that the BLE connection is established at step S112, the portable electrocardiograph 10 transmits a report indicating that the measurement is ended to the smartphone 20 (S113).

The smartphone 20 having received the measurement end report transmits a BLE communication end request to the portable electrocardiograph 10 (S209) to disconnect the BLE connection (S210), and ends the series of processing steps on the smartphone 20 side. Various types of information received by the smartphone 20, such as the electrode contact state and the electrocardiogram data, can be stored in the storage unit 24 and used as appropriate. The portable electrocardiograph 10 having received the communication end request of step S209 from the smartphone 20 disconnects the BLE connection (S114) and ends the series of processing steps.

According to the portable electrocardiograph 10 and the biological information measurement system 1 described in the present embodiment, the contact state of the first right electrode 12b and the left electrode 12a with respect to the skin can be automatically detected, and the contact state levels can be classified into stages and indicated to the user before and during the electrocardiographic measurement. By linking with the information processing terminal such as the smartphone 20, various types of information can be displayed on the display and viewed, such as the advice on how to make good contact with the electrodes, the electrocardiographic waveform data, and the like, as well as the contact state level. Thus, the user can intuitively recognize how much the electrode should be pressed against the skin, whereby the electrocardiographic waveform can be acquired with good contact state and low myoelectric noise. The data received by the smartphone 20 can be saved and effectively utilized by using the application recording medium or the like.

Since the portable electrocardiograph 10 can measure and save the electrocardiographic waveform, detect, classify, and save the electrode contact level, analyze the electrocardiographic waveform data, and display and save the analysis results independently of the smartphone 20, the electrocardiographic measurement can be performed at a desired timing even when the communication connection with the smartphone 20 cannot be established.

### Modified Example

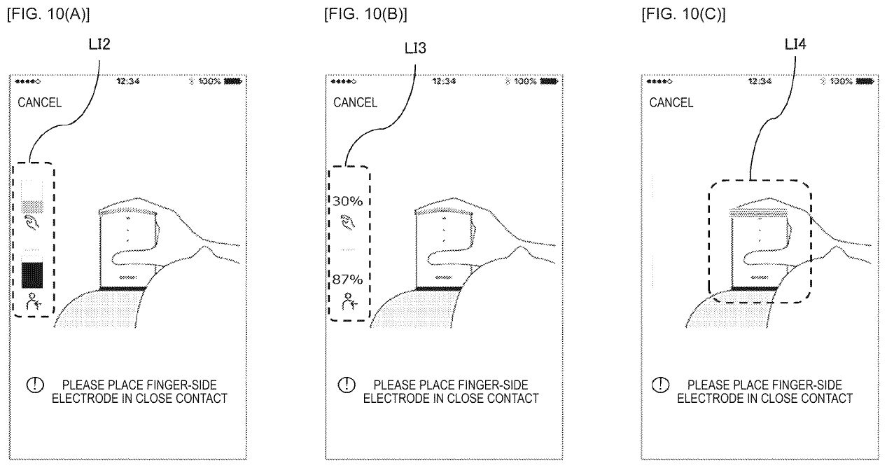

The above embodiment has illustrated the example of classifying the contact levels of each electrode with respect to the skin into four levels, but the contact state classification unit 113 may classify the electrode contact levels more finely or into three levels, i.e., less than four levels. The electrode contact level indicator LI is not limited to activation of the display area of the plurality of (divided) display segments, and various display modes can be employed. FIGS. 10(A) to 10(C) illustrate modified examples of such an electrode contact level indicator LI. An electrode contact level indicator LI2 illustrated in FIG. 10(A) indicates the electrode contact level by the size of the area on a consecutive bar whose display is activated for each of the first right electrode 12b and the left electrode 12a.

An electrode contact level indicator LI3 illustrated in FIG. 10(B) is an example of illustrating the degree of contact in numerical values (in percentage) for each of the first right electrode 12b and the left electrode 12a. An electrode contact level indicator LI4 illustrated in FIG. 10(C) is an example of indicating the electrode contact state by color and its transparency for each of the first right electrode 12b and the left electrode 12a. For example, the display mode may be such that the transparency decreases and the color becomes darker as the contact level is higher or the transparency decreases and the color becomes darker as the same contact level continues, and the contact level is definitively indicated when the transparency reaches zero.

In the embodiment described above, the information such as the electrode contact state, the electrocardiographic measurement time, and the like and the electrocardiographic signal (waveform data) may be transmitted and received by different transmission and reception methods. Specifically, the information of a relatively small data capacity, such as the electrode contact state and the electrocardiographic measurement time, may be transmitted and received in streaming format, and the electrocardiographic waveform data having a large data capacity may be transmitted and received via high-speed data communication.

### Second Embodiment

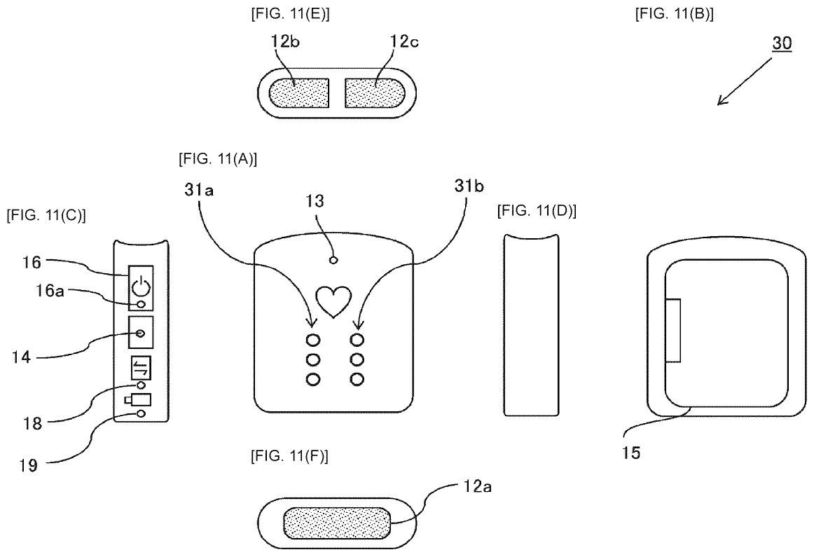

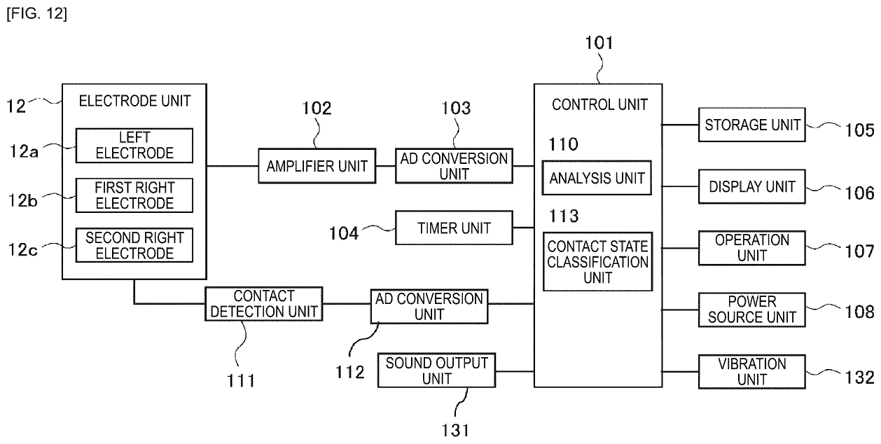

With reference to FIGS. 11(A) to 13, a second embodiment of the present invention will be described. FIG. 11(A) to 11(F) illustrate the configuration of a portable electrocardiograph 30 according to the present embodiment. FIG. 11(A) is a front view illustrating the front of the body. Similarly, FIG. 11(B) is a rear view, FIG. 11(C) is a left side view, FIG. 11(D) is a right side view, FIG. 11(E) is a plan view, and FIG. 11(F) is a bottom view. FIG. 12 is a block diagram illustrating a functional configuration of the portable electrocardiograph 30. For the most part, the portable electrocardiograph 30 according to the present embodiment has a configuration similar to that of the portable electrocardiograph 10 of first embodiment, so that the same reference signs will be used for the same constituent components and repeated descriptions thereof will be omitted.

### Device Configuration

The portable electrocardiograph 30 is designed not to communicate with other devices and, in this respect, has a different configuration from the portable electrocardiograph 10. Specifically, as illustrated in FIG. 11(C), the electrocardiograph 30 is provided with an analysis result reporting LED 14 on the left side surface thereof, instead of the BLE communication button 17 and the BLE communication LED 17a. As illustrated in FIG. 11(A), the electrocardiograph 30 is provided with a left electrode contact level indicator LED 31a and a right electrode contact level indicator LED 31b in addition to the measurement state reporting LED 13 on the front surface thereof. These are all equipped with three LED indicator lights, and the number of LED indicator lights being lit can indicate to the user the contact state level of each electrode.

As illustrated in FIG. 12, the portable electrocardiograph 30 does not include the communication unit 109 as compared with the portable electrocardiograph 10, but is provided with functional units including a sound output unit 131 and a vibration unit 132. The sound output unit 131 includes a speaker not illustrated, and can report the information such as the contact state level classified by the contact state classification unit 113 by sound. The vibration unit 132 includes a vibrator not illustrated, and can report the information such as the contact state level classified by the contact state classification unit 113 by vibration (and by pattern). The other parts are the same as those of the portable electrocardiograph 10 including the electric circuit configuration for detecting the potential difference between the electrodes.

### Measurement Processing Flow

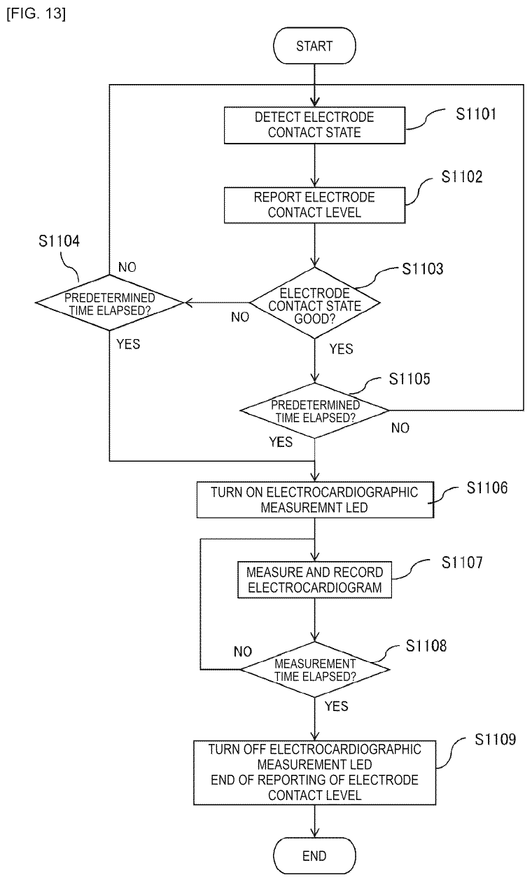

The measurement processing flow by the portable electrocardiograph 30 will be described with reference to FIG. 13. FIG. 13 is a flowchart illustrating an example processing flow according to electrocardiographic measurement by the portable electrocardiograph 30.

Prior to the measurement, the user operates the power source switch 16 to turn on the power source of the portable electrocardiograph 30. Accordingly, the power source LED is lit to indicate that the power source is turned on. The user holds the portable electrocardiograph 30 with the right hand, and places the right index finger in contact with the first right electrode 12b and the second right electrode 12c, while placing the left electrode 12a in contact with the skin at a location to be measured. The control unit 101 detects the contact state of each electrode with respect to the skin via the contact detection unit 111 and the AD conversion unit 112 (S1101).

Subsequently, the control unit 101 (the contact state classification unit 113) classifies the detected contact state into four stages of “good”, “slightly poor contact”, “poor contact”, and “no contact”, and reports the classified contact level to the user (S1102). Specifically, the contact states of the left electrode 12a and the first right electrode 12b are respectively indicated by the number of lights of each of the three LED indicator lights of the left electrode contact level indicator LED 31a and the right electrode contact level indicator LED 31b. For example, the number of lights of the indicator lamp can be set to zero in the case of “no contact”, the number of lights of the indicator lamp can be set to 1 in the case of “poor contact”, the number of lights of the indicator lamp can be set to 2 in the case of “slightly poor contact”, and the number of lights of the indicator lamp can be set to 3 in the case of “good contact”.

Subsequently, the control unit 101 performs processing of determining whether the contact states of both of the first right electrodes 12b and the first left electrodes 12a are “good” (S1103). If it is determined here that the contact state of at least one of the electrodes is not “good”, the process proceeds to step S1104 in which it is determined whether predetermined time has elapsed in that state (S1104). The predetermined time here is set to a reasonable amount of time (for example, five seconds) for waiting to allow the user to make the electrode contact state be “good”.

If it is determined in step S1104 that the predetermined time has not elapsed, the control unit 101 returns the process to step S1011 and the subsequent processing steps are repeated. If it is determined that the predetermined time has elapsed in step S1104, the control unit 101 causes the process to proceed to step S1106.

If the control unit 101 determines that the contact states of all electrodes are “good” in step S1103, processing of determining whether the predetermined time has elapsed in that state is performed (S1105). The predetermined time here is set to a reasonable amount of time (for example, three seconds) for determining whether the “good” contact state is stable rather than transient for both electrodes. If it is determined in step S1105 that the predetermined time has not elapsed, the control unit 101 returns to step S1101 and the subsequent processing steps are repeated. If it is determined that the measurement time has elapsed in step S1105, the process proceeds to step S1106.

When executing the electrocardiographic measurement, the control unit 101 indicates that electrocardiographic measurement is in progress by blinking the measurement state reporting LED 13 on the front surface of the main body at a predetermined rhythm (S1106), and stores the electrocardiographic signal output from the differential amplifier 94 and acquired via the AD conversion unit 103 in the storage unit 105 as needed (S1107). Here, the classified electrode contact state level is also stored in the storage unit together with the electrocardiographic signal. The stored electrocardiographic signal and electrode contact state level information are tied to the time information at which the respective information is acquired and stored in the storage unit 105.

In step S1108, the control unit 101 executes processing of determining whether predetermined time (for example, 30 seconds) for the electrocardiographic measurement has elapsed (S1108). Here, if it is determined that the predetermined time has not elapsed, the process returns to step S1107, and the subsequent processing steps are repeated. If it is determined in step S1108 that the predetermined measurement time has elapsed, the measurement state reporting LED 13 is turned off, and the reporting of the contact level is ended (S1109), thereby ending the series of processing steps related to the electrocardiographic measurement.

According to the portable electrocardiograph 30 of the present embodiment as described above, the electrocardiograph can report the contact state level of the electrode in each stage, execute the electrocardiographic measurement processing, and record the electrocardiographic waveform data by itself. Since the level of the classified contact state is also stored along with the electrocardiographic waveform data, when the stored electrocardiographic waveform is later checked by, for example, displaying it on a display, the contact state levels of the first right electrode 12b and the left electrode 12a when the waveform is detected can also be checked along with the electrocardiographic waveform.

### Modified Example

Although the measurement processing flow according to the second embodiment has described only the example of reporting the contact state of the electrode with respect to the skin by the left electrode contact level indicator LED 31a and the right electrode contact level indicator LED 31b, the contact state can be reported by sound or vibration in addition to or instead of the above. In this case, since continuous vibration or continuous sound output during the electrocardiographic measurement may interfere with the measurement, the report may be provided by the sound or vibration only before the measurement processing.

The portable electrocardiograph can be made to display various types of information using a liquid crystal display or other display means, instead of using various LED indicator lights, such as the left electrode contact level indicator LED 31a and the right electrode contact level indicator LED 31b. In such a case, the electrode contact level may be reported in the display mode as described in the first embodiment and the modified example thereof.

### Supplemental Note

The description of each example described above has been provided as merely illustrative of the present invention, and the present invention is not limited to the specific forms described above. Various modifications and combinations may be made within the scope of the technical idea of the present invention. For example, the portable electrocardiograph illustrated in the second embodiment may be provided with a communication unit to allow communication with the information processing terminal. In this way, the electrocardiograph can report the contact level to the user by itself, and by linking it with the information processing terminal, it is possible to report the contact level with higher usability.

The communication processing unit may not be limited to the communication unit intended for the BLE communication, and may be an antenna capable of other wireless communications such as Wi-Fi (trade name) or infrared communication. Alternatively, the wired connection may be used to connect to other information processing terminals. Although the present invention has been employed in the portable electrocardiograph in the above description, the present invention is also applicable to a non-portable electrocardiograph or other biological information measurement devices other than the electrocardiograph.

The information processing terminals are not limited to the smartphones, and may be other portable information processing terminals such as a tablet terminal or a stationary terminal.

### REFERENCE SIGNS LIST

1 Biological information measurement system
10, 30 Portable electrocardiograph
13 Measurement state reporting LED
14 Analysis result reporting LED
15 Battery cover
16 Power source switch
16a Power source LED
17 Communication button
17a BLE communication LED
18 Memory residual indicator LED
19 Battery change LED
31a Left electrode contact level indicator LED
31b Right electrode contact level indicator LED
911 Right pull-up resistor
912 Right non-inverting amplifier
913 Right buffer amplifier
915 Right contact state signal
921 Left pull-up resistor
922 Left non-inverting amplifier
923 Left buffer amplifier
925 Left contact state signal
931 First amplification factor determining resistor
932 Second amplification factor determining resistor
933 Third amplification factor determining resistor
94 Differential amplifier
941 Electrocardiographic signal
GND Reference potential
V1 Power source potential
LI, LI2, LI3, LI4 Electrode contact level indicator

1. A biological information measurement device provided with a first electrode, a second electrode, and a third electrode, and configured to measure biological information of a measurement target based on a potential difference between the first electrode and the second electrode with a potential of the third electrode as a reference potential, the biological information measurement device comprising:
a first converter configured to output a signal related to a contact state of the first electrode with respect to the measurement target based on a potential of the first electrode that varies depending on a contact state of the first electrode with respect to the measurement target;
a second converter configured to output a signal related to a contact state of the second electrode with respect to the measurement target based on a potential of the second electrode that varies depending on a contact state of the second electrode with respect to the measurement target;
a contact state classification unit configured to classify a level of the contact state of each of the first electrode and the second electrode with respect to the measurement target into at least three stages based on a signal output from each of the first contact signal output unit and the second contact signal output unit;
a reporting device configured to report the contact state of each of the first electrode and the second electrode with respect to the measurement target in a manner that the level classified by the contact state classification unit is identifiable;
a display configured to display guidance information for making a good state of contact of the first electrode and the second electrode according to the level of respective contact states of the first electrode and the second electrode with respect to the measurement target; and
a processor configured to execute processing of measuring the biological information.

2. The biological information measurement device according to claim 1, wherein the reporting device includes a sound output that reports by sound.

3. The biological information measurement device according to claim 1, wherein the reporting device includes a vibration device that reports by vibration.

4. The biological information measurement device according to claim 1, wherein the reporting device includes a display that reports by display.

5. The biological information measurement device according to claim 4, wherein the reporting device indicates the level by displaying at least one of a numerical value, a number of a plurality of display segments whose display is activated, a size of an area whose display is activated, or a change in color and transparency of a display area, in the display.

6. The biological information measurement device according to claim 1, wherein the reporting device reports the contact state of each of the first electrode and the second electrode with respect to the measurement target before and/or during the measurement processing of the biological information.

7. A biological information measurement system, comprising:
a biological information measurement device provided with a first electrode, a second electrode, and a third electrode, and configured to measure biological information of a measurement target based on a potential difference between the first electrode and the second electrode with a potential of the third electrode as a reference potential; and
an information processing terminal configured to communicate with the biological information measurement device, wherein
the biological information measurement device includes
a processor configured to execute processing of measuring the biological information,
a first converter configured to output a signal related to a contact state of the first electrode with respect to the measurement target based on a potential of the first electrode that varies depending on a contact state of the first electrode with respect to the measurement target, and
a second converter configured to output a signal related to a contact state of the second electrode with respect to the measurement target based on a potential of the second electrode that varies depending on a contact state of the second electrode with respect to the measurement target,
at least one of the biological information measurement device or the information processing terminal includes
a contact state classification unit configured to classify a level of the contact state of each of the first electrode and the second electrode with respect to the measurement target into at least three stages based on a signal output from each of the first contact signal output unit and the second contact signal output unit, and
the information processing terminal includes
a reporting device configured to report the contact state of each of the first electrode and the second electrode with respect to the measurement target in a manner that the level classified by the contact state classification unit is identifiable, and
a display configured to display guidance information for making a good state of contact of the first electrode and the second electrode according to the level of respective contact states of the first electrode and the second electrode with respect to the measurement target.

8. The biological information measurement system according to claim 7, wherein the reporting device includes a display that reports by display.

9. The biological information measurement system according to claim 8, wherein the reporting device indicates the level by displaying at least one of a numerical value, a number of a plurality of display segments whose display is activated, a size of an area whose display is activated, or a change in color and transparency of a display area, in the display.

10. The biological information measurement system according to claim 7, wherein the reporting device reports the contact state of each of the first electrode and the second electrode with respect to the measurement target before and/or during the measurement processing of the biological information.
